(19) United States
(12) Patent Application Publication
(10) Pub. No.: US 2009/0069603 A1
(43) Pub. Date: Mar. 12, 2009

(54) PROCESS FOR HYDROGENATING NITRILES TO PRIMARY AMINES OR AMINONITRILES AND CATALYSTS SUITABLE THEREFOR

(75) Inventors: Thilo Hahn, Kirchheimbolanden (DE); Martin Ernst, Heidelberg (DE); Johann-Peter Melder, Bohl-Iggelheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/067,096

(22) PCT Filed: Sep. 11, 2006
(86) PCT No.: PCT/EP2006/066228

§ 371 Date: Jul. 15, 2008

(30) Foreign Application Priority Data
Sep. 15, 2005 (DE) 102005044187.4
Oct. 11, 2005 (DE) 102005048999.0
Nov. 29, 2005 (DE) 102005057198.0

(51) Int. Cl. C07C 209/00; B01J 23/42

(52) U.S. Cl. 564/491; 502/176; 502/174; 502/157; 502/152; 502/213; 502/171

(57) ABSTRACT

The invention relates to a process for hydrogenating oligonitriles which have at least two nitrile groups in the presence of a catalyst which, before commencement of the hydrogenation, is pretreated by contacting with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formiates, alkaline earth metal formiates, ammonium formiate, alkali metal oxalates, alkaline earth metal oxalates and ammonium oxalate.

The invention relates to a process for hydrogenating oligonitriles which have at least two nitrile groups in the presence of a catalyst which, before commencement of the hydrogenation, is pretreated by contacting with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formates, alkaline earth metal formates, ammonium formate, alkali metal oxalates, alkaline earth metal oxalates and ammonium oxalate.

The invention also relates to oligoamines or aminonitriles obtainable from oligonitriles by this process, and to the use of catalysts as defined at the outset for full or partial hydrogenation of oligonitriles.

The invention further relates to a catalyst comprising a metal from groups 8 to 10 of the Periodic Table which, before use, is pretreated with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formates, alkaline earth metal formates, ammonium formate, alkali metal oxalates, alkaline earth metal oxalates and ammonium oxalate, excluding cobalt or nickel catalysts pretreated with alkali metal carbonates or alkali metal hydrogencarbonates.

The invention finally relates to a process for preparing this catalyst, which comprises treating a metal from groups 8 to 10 of the Periodic Table with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formates, alkaline earth metal formates, ammonium formate, alkali metal oxalates, alkaline earth metal oxalates and ammonium oxalate, excluding processes for preparing cobalt or nickel catalysts pretreated with alkali metal carbonates or alkali metal hydrogencarbonates.

Amines having at least-two amino groups and aminonitriles have various uses and, apart from in solvents, crop protection compositions, surfactants and pharmaceuticals, they are used especially as a starting material for polyamides. They are generally prepared by hydrogenating nitrites.

Nitriles having more than one nitrile group —CN in the molecule are referred to hereinbelow as oligonitriles. When all nitrile groups present in the molecule are hydrogenated, which is referred to below as full hydrogenation, oligoamines are obtained. When not all, but rather only some of the nitrile groups present in the molecule are hydrogenated, referred to below as partial hydrogenation, aminonitriles are obtained. In schematic terms:

R—(CN)ₙ—^{(a)}→(H₂N)ₓ—R—(CN)_{y}—^{(b)}→R—(NH₂)ₙ (1)
where (a) is partial and (b) full hydrogenation, and
R means organic radical,
n means integer from 2 to 20,
x, y mean integer ≧1, where x+y=n.

For example, partial hydrogenation of adiponitrile (ADN) affords aminocapronitrile (ACN) which is processed further to give caprolactam which is polymerized to give nylon-6. Full hydrogenation affords hexamethylenediamine (HMD) which is used for nylon-6,6 preparation.

The hydrogenation is undertaken typically with hydrogen over nickel or cobalt catalysts which are preferably present in the form of metal sponge, for example in the form of Raney® nickel or Raney® cobalt. Partial and full hydrogenation generally proceed in succession and a random mixture of aminonitriles, oligoamines and other by-products is obtained, for example a mixture of aminonitrile (ACN) and diamine (HMD) and also by-products in the hydrogenation of dinitriles (ADN).

The suppression of full hydrogenation or the establishment of a desired nonrandom aminonitrile/oligoamine ratio is possible by virtue of specific configurations of the hydrogenation, for example catalyst doping with noble metals or additional use of fluorides or cyanides. Such processes for partial hydrogenation are described, for example, in U.S. Pat. No. 5,151,543, WO 99/47492, U.S. Pat. No. 5,981,790, WO 00/64862, WO 01/66511, and WO 03/000651. One possibility is the pretreatment or conditioning of the hydrogenation catalyst.

For example, said WO 01/66511 describes the hydrogenation of nitrile groups to amino groups, for example the hydrogenation of dinitriles to aminonitriles or diamines, with hydrogen over a hydrogenation catalyst (e.g. Raney® nickel or cobalt) which is conditioned beforehand. The conditioning is effected by mixing the catalyst with a strong mineral base (e.g. hydroxides of the alkali metals or alkaline earth metals) in a solvent in which the base is sparingly soluble.

DE 102 07 926 A1 describes the preparation of primary amines by hydrogenation of nitrites, in which the nitrile, hydrogen and, if appropriate, ammonia are converted over a cobalt or nickel catalyst. The catalyst is modified ex situ (before the hydrogenation reaction) by adsorption of an alkali metal carbonate or hydrogencarbonate. Preference is given to nitrites of the formula R—CN where R=saturated or unsaturated hydrocarbon group. There is no mention of the hydrogenation of dinitriles or other oligonitriles nor of the possibility of a partial instead of full hydrogenation; in the examples, only mononitriles are hydrogenated: lauronitrile to dodecylamine or oleylnitrile to oleylamine.

The known processes have at least one of the following disadvantages:
the aminonitrile/oligoamine ratio, i.e. the ratio of partial to full hydrogenation, has poor controllability,
the selectivity in a partial hydrogenation is low: instead of the desired aminonitriles, hydrogenation proceeds fully to the oligoamines,
large amounts of by-products are obtained and are difficult to remove,
toxic substances are used additionally and have to be removed in a costly and inconvenient manner and disposed of separately,
the noble metal doping makes the catalyst more expensive,
the hydrogenation of mononitriles cannot be applied directly to the hydrogenation of dinitriles.

It was an object of the invention to remedy the disadvantages outlined. The intention was to provide a process for hydrogenating nitrites having at least two nitrile groups, with which it is possible to prepare amines or aminonitriles, i.e. the process was to enable full or partial hydrogenation. In particular, the possibility was to exist of keeping the extent of full hydrogenation low.

Moreover, the intention was that a low level of by-products would occur. The process was not to need any toxic substances, for example cyanides, and to be operable without expensive noble metal doping of the catalyst.

Accordingly, the hydrogenation process specified at the outset has been found. Also found have been the oligoamines and aminonitriles obtainable therewith, and also the use of the catalysts for full or partial hydrogenation of oligonitriles. Additionally found has been the catalyst defined at the outset, and also a process for its preparation. The preferred embodiments of the invention can be taken from the subclaims. All pressures specified below are absolute pressures.

Suitable oligonitriles which can be used in the hydrogenation process according to the invention are adiponitrile (ADN), succinonitrile, iminodiacetonitrile, suberonitrile or iminodipropionitrile (bis[cyanoethyl]amine). Likewise useful are aromatic amines such as m-xylylenediamine or ortho-, meta- or para-phthalnitrile. Suitable oligonitriles having at least three nitrile groups are, for example, nitrilotrisacetonitrile (tris[cyanomethyl]amine), nitrilotrispropionitrile (tris[cyanoethyl]amine), 1,3,6-tricyanohexane or 1,2,4-tricyanobutane.

Preferred oligonitriles are those having two nitrile groups. Particularly preferred dinitriles are those having terminal nitrile groups, i.e. alpha,omega-dinitriles. Very particular preference is given to using adiponitrile.

In a preferred embodiment referred to here as full hydrogenation, in the process, all nitrile groups present in the nitrile molecule are hydrogenated to amino groups (full hydrogenation) to form an oligoamine. This oligoamine no longer comprises any nitrile groups.

Preference is given to hydrogenating an alpha,omega-dinitrile by full hydrogenation to give an alpha,omega-diamine. In particular, adiponitrile (ADN) is hydrogenated to hexamethylenediamine (HMD).

In an equally preferred embodiment referred to here as partial hydrogenation, in the process, only some of the nitrile groups present in the nitrile molecule are hydrogenated to amino groups (partial hydrogenation) to obtain an aminonitrile.

In the partial hydrogenation of oligonitriles having three nitrile groups, it is possible to obtain a diaminomononitrile or a monoaminodinitrile depending on whether one or two of the three nitrile groups are hydrogenated to the amino group.

Preference is given to hydrogenating an alpha,omega-dinitrile by partial hydrogenation to give an alpha,omega-aminonitrile. In particular, adiponitrile is hydrogenated to give aminocapronitrile (ACN).

In the hydrogenation, the oligonitrile is reacted with hydrogen or a hydrogen-comprising gas over the catalyst (see below). The hydrogenation can be carried out, for example, in suspension (suspension hydrogenation), or else over a fixed, moving or fluidized bed, for example over a fixed bed or over a fluidized bed. These embodiments are known to those skilled in the art.

In general, hydrogen gas or a mixture of hydrogen and an inert gas such as nitrogen or argon is used. Alternatively, and depending upon the pressure and temperature conditions established, the hydrogen or the mixture may also be present in dissolved form. When full hydrogenation is desired, the hydrogen may be used in excess; in the case of partial hydrogenation, the amount of hydrogen required in stoichiometric terms for this purpose can be metered in.

The amount of catalyst in the suspension hydrogenation is generally from 1 to 30% by weight, preferably from 5 to 25% by weight, based on the contents of the hydrogenation reactor. In the case of supported catalysts, the support material is included in the calculation. When hydrogenation is effected over a fixed bed or a fluidized bed, the amount of catalyst, if appropriate, has to be adjusted in a customary manner.

The hydrogenation is preferably carried out in liquid phase. The reaction mixture comprises typically at least one solvent; suitable examples are amines, alcohols, ethers, amides or hydrocarbons. The solvent preferably corresponds to the reaction product to be prepared, i.e. an oligoamine or aminonitrile is used as the solvent.

Suitable amines are, for example, hexamethylenediamine or ethylenediamine. Suitable alcohols are preferably those having from 1 to 4 carbon atoms, for example methanol or ethanol. Suitable ethers are, for example, methyl tert-butyl ether (MTBE) or tetrahydrofuran (THF). Useful amides are, for example, those having from 1 to 6 carbon atoms. Suitable hydrocarbons are, for example, alkanes such as the hexanes or cyclohexane, and also aromatics, for example toluene or the xylenes.

The amount of solvent in the reaction mixture is typically from 0 to 90% by weight. If solvent and product are identical, the amount of solvents may be over 99% by weight.

In the case of a full hydrogenation, the reaction can also be carried out in the absence of an additional solvent in product mode, for example in the case of the full hydrogenation of ADN in HMD.

Preference is likewise given to carrying out the hydrogenation without addition of water. Water present in the feedstocks, for example as an impurity or in the Raney® catalyst to prevent self-ignition, can be removed beforehand.

In the hydrogenation, it is possible to additionally use ammonia or another base, for example alkali metal hydroxides, for example in aqueous solution. If this is the case, the amount of ammonia or of the base is generally from 1 to 10% by weight, based on the oligonitrile. Ammonia is also suitable as a solvent.

The reaction temperature is typically from 30 to 250° C., preferably from 50 to 150° C. and in particular from 60 to 110° C. The pressure is typically from 1 to 300 bar, preferably from 2 to 160 bar, in particular from 2 to 85 bar and more preferably from 5 to 35 bar.

The process can be operated continuously, semicontinuously (semibatchwise) or discontinuously (batchwise), for which all reactor types common for hydrogenation reactions are suitable. The reaction mixture is worked up to the product (diamine or aminonitrile) in a customary manner, for example by distillation.

Whether the hydrogenation proceeds as a full or partial hydrogenation and in what ratio oligoamines (full hydrogenation) and aminonitriles (partial hydrogenation) are present in the resulting reaction mixture depends upon factors including reaction temperature, pressure and time, upon composition and amount of the catalyst, upon type and amount of the oligonitrile, upon the amount of hydrogen, and upon the type and amount of any additives additionally used, such as ammonia or other bases.

Typically, a lower reaction temperature, a lower pressure, a lower amount of hydrogen and especially a shorter reaction time favor partial over full hydrogenation.

The designation of the element groups in the Periodic Table of the Elements (PTE) used below corresponds to the new IUPAC system, i.e. the groups are numbered serially from 1=hydrogen and alkali metals to 18=noble gases. See, for example, inside front cover in the CRC Handbook of Chemistry and Physics, 86th edition 2005, CRC Press/Taylor & Francis, Boca Raton, Fla., USA.

The catalyst comprises preferably at least one metal M from groups 8 to 10 of the Periodic Table (Fe, Ru, Os, Co, Rh, Ir, Ni, Pd, Pt). As the metal M, it preferably comprises iron, cobalt, nickel or mixtures thereof. Particular preference is given to cobalt and nickel, especially nickel. The metals mentioned are preferably present in the oxidation state zero, but may also have other oxidation states.

Metal sponge catalysts, for example those according to Raney®, are particularly preferred. In the process, the catalyst is preferably a nickel sponge catalyst or a cobalt sponge catalyst (each Raney®). To prepare these highly active catalysts particularly suitable for hydrogenations, nickel or cobalt is typically alloyed with Al, Si, Mg or Zn metal, frequently with Al, and the alloy is comminuted and the metal other than nickel or cobalt is leached out with alkalis. This leaves a skeleton-like metal sponge, known as Raney® nickel or Raney® cobalt. Raney® catalysts are also commercially available, for example from Grace.

In order to prevent self-ignition of the pyrophoric Raney® catalysts, they are kept moist with, for example, water. Before the catalyst is used in the inventive hydrogenation, this water can be removed.

In addition to the metals M mentioned, the catalyst may comprise at least one further metal D which is selected from groups 1 to 7 of the Periodic Table. The further metals D are also referred to as doping metals or promoters. The doping allows the activity and selectivity of the catalyst to be varied as required.

As the further metal D, the catalyst preferably comprises at least one of the metals titanium, zirconium, chromium, molybdenum, tungsten and manganese. The amount of a single further metal D is typically from 0 to 15% by weight, preferably from 0 to 10% by weight, based on the metal M.

The catalyst may be present as such, for example as pure metal or alloy, in the form of fine particles or as metal sponge (Raney®). It is equally possible to use it in supported form. Suitable supports are inorganic support materials such as alumina, magnesia or silica, and also carbon. Also suitable are supports comprising catalytically active metal oxides or those active as a dopant, for example zirconium dioxide, manganese(II) oxide, zinc oxide or chromium(VI) oxide.

Supported catalysts can be prepared in a customary manner, for example by impregnation, coprecipitation, ion exchange or other processes. In the case of supported catalysts, the support makes up typically from 20 to 99% by weight, preferably from 50 to 90% by weight, of the supported catalyst.

According to the invention, the catalyst, before commencement of the hydrogenation, is pretreated by contacting with a compound A. The compound A is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formates, alkaline earth metal formates, ammonium formate, alkali metal oxalates, alkaline earth metal oxalates and ammonium oxalate.

According to the invention, the compounds A also include the hydrates (for example those which comprise the water as water of constitution and/or those which comprise the water as water of crystallization) and the carbonates, which may be basic, of the aforementioned compounds or compound classes A. The basic alkaline earth metal carbonates or oxocarbonates also include, for example, basic magnesium carbonate Mg(OH)₂·4MgCO₃·4H₂O.

The catalyst comprises preferably from 0.01 to 25% by weight, in particular from 0.5 to 15% by weight and more preferably from 1 to 10% by weight of alkali metal, alkaline earth metal or ammonium, based on the pretreated catalyst. In this case, alkali metal or alkaline earth metal or ammonium are counted as such, i.e. without carbonate, hydrogencarbonate, oxocarbonate, carboxylate, dihydrogenphosphate, hydrogenphosphate or phosphate radical, and the support material is included in the calculation in the case of supported catalysts.

The alkali metal in compound A is preferably selected from lithium, sodium and potassium, in particular sodium or potassium. The alkaline earth metal is preferably selected from magnesium and calcium. It is evident from the examples which have since become available that this preference is unfounded. Omit?

In particular, the compound A used is sodium carbonate, potassium carbonate, magnesium carbonate, calcium carbonate, ammonium carbonate, sodium hydrogencarbonate, potassium hydrogencarbonate, magnesium hydrogencarbonate, calcium hydrogencarbonate, ammonium hydrogencarbonate, magnesium oxocarbonate or mixtures thereof.

For the alkali metals, preference is likewise given to phosphates and hydrogenphosphates.

The carboxylates are preferably selected from the formates, acetates, propionates, butanoates, pentanoates, hexanoates, as well as dicarboxylates such as oxalates, malonates and succinates, glutarates and adipates. When the corresponding acids are discussed what is meant is, for example, formic acid, acetic acid, propionic acid, butyric acid, pentanoic acid, hexanoic acid, oxalic acid, malonic acid, succinic acid, glutaric acid and adipic acid.

It is also possible to use a mixture comprising alkali metal and alkaline earth metal compounds. In this mixture, the proportion of alkaline earth metal compounds is, for example, from 1 to 99% by weight.

The catalyst can be pretreated outside the reactor used for the hydrogenation, or in the hydrogenation reactor before commencement of the actual hydrogenation. It is equally possible to pretreat a catalyst which has already been used beforehand in a hydrogenation, i.e. spent catalyst can be regenerated by contacting with the compound A.

In a preferred embodiment of the hydrogenation process, the catalyst is pretreated by contacting it with a solution or suspension of compound A.

The preferred solvent or suspension medium is water, but the organic solvents already mentioned above for the hydrogenation are also suitable. Compound A can be used in solid form and the corresponding solution or suspension can be prepared by adding the solvent or suspension medium. The content of compound A in such an aqueous or nonaqueous solution or suspension is typically from 1 to 90% by weight.

Especially in a suspension hydrogenation, the contacting can be effected in a simple manner by slurrying the catalyst in the solution or suspension of compound A, in which case the amount of catalyst is appropriately from 5 to 95% by weight, based on the solution or suspension of the compound A. Subsequently, the excess solution or suspension can be removed, for example by decantation or filtration.

It is advantageous to wash the catalyst thereafter once or more than once with one or more different organic liquids in order to remove adhering water. For example, the pretreated, filtered-off or decanted catalyst can be washed first once or more than once with an alcohol such as methanol or ethanol, and then with a hydrocarbon, for example cyclohexane, or with an ether.

Accordingly, in the hydrogenation process, the catalyst is preferably contacted with an aqueous solution or suspension of compound A, the catalyst is removed and it is subsequently washed with at least one organic liquid in order to remove the water.

The contacting (slurrying), removal (filtration, decantation) and washing are effected appropriately under inert gas. Pressure and temperature for the contacting are generally not critical. For example, it is possible to work at room temperature (20° C.) and ambient pressure.

The duration of the contacting depends, for example, upon the desired content of compound A in the catalyst, and especially the adsorption behavior of the catalyst, its outer and inner surface area and the catalyst support material used if appropriate. It is, for example, from 5 min to 5 hours, preferably from 10 min to 2 hours.

Alternatively, the contacting can be configured such that the compound A is formed in situ before or during the treatment of the catalyst. To this end, a suspension or solution of catalyst, water or another suspension medium or solvent already mentioned above and a compound A* is prepared, and carbon dioxide or the corresponding carboxylic acid or phosphoric acid is introduced into this suspension or solution. In the case of carbonates, hydrogencarbonates and oxocarbonates compound A* is an alkali metal, alkaline earth metal or ammonium compound other than the compounds A. In the case of carboxylates and phosphates, they may be the carbonates, hydrogencarbonates or oxocarbonates of alkali metals, alkaline earth metals or ammonium and the hydroxides.

Compound A* preferably comprises water-soluble salts, for example the halides, nitrates or sulfates of alkali metals, alkaline earth metals or ammonium. Reaction with the CO₂ introduced or the acid fed in forms the desired carbonates, hydrogencarbonates or oxocarbonates or carboxylates, dihydrogenphosphates, hydrogenphosphates or phosphates A from compound A*. The reaction with the CO₂ or the acid can be effected, for example, at room temperature and ambient pressure.

Consequently, in this embodiment of the process, compound A is formed in situ by introducing carbon dioxide or a carboxylic acid or phosphoric acid into a suspension or solution which the catalyst and a compound A* which, in the case of carbonates, hydrogencarbonates and oxocarbonates, is an alkali metal, alkaline earth metal or ammonium compound different from compounds A.

The catalyst can also be contacted in another way, for instance by mixing the untreated catalyst with solid compound A, by drum application of solid compound A onto the untreated catalyst, or by spraying the untreated catalyst with a solution or suspension of compound A.

The catalyst pretreated with compound A is dried, i.e. any solvent or suspension medium used is removed, in a customary manner. Alternatively, the catalyst may also be used in moist or suspended form; for example, the pretreated catalyst, after the washing with the organic liquid, can be left in the wash liquid used last and this suspension can be used.

The oligoamines or aminonitriles obtainable by the hydrogenation process according to the invention likewise form part of the subject matter of the invention.

The invention further provides for the use of catalysts as described above for full or partial hydrogenation of oligonitriles. Preference is given to the use of the catalysts for full hydrogenation of alpha,omega-dinitriles to alpha,omega-diamines. It is particularly preferred that the catalyst is used for full hydrogenation of adiponitrile to hexamethylenediamine.

Preference is likewise given to the use of the catalysts for partial hydrogenation of alpha,omega-dinitriles to alpha,omega-aminonitriles. It is particularly preferred that the catalyst is used for partial hydrogenation of adiponitrile to aminocapronitrile.

The invention further provides a catalyst comprising a metal from groups 8 to 10 of the Periodic Table which, before use, is pretreated with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formates, alkaline earth metal formates, ammonium formate, alkali metal oxalates, alkaline earth metal oxalates and ammonium oxalate. In delimitation from said DE 102 07 926 A1, cobalt or nickel catalysts pretreated with alkali metal carbonates or alkali metal hydrogencarbonates are excluded.

The catalyst preferably has at least one of the features specified above in the description of the catalyst, especially at least one of the features from claims 9 to 19.

Finally, a process for preparing this catalyst also forms part of the subject matter of the invention. This process comprises treating a metal from groups 8 to 10 of the Periodic Table with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formates, alkaline earth metal formates, ammonium formate, alkali metal oxalates, alkaline earth metal oxalates and ammonium oxalate, again excluding processes for preparing cobalt or nickel catalysts pretreated with alkali metal carbonates or alkali metal hydrogencarbonates.

This catalyst preparation process preferably has at least one of the features specified above in the description of the catalyst preparation. In particular, it has at least one of the features from claims 18 to 20.

It is possible with the hydrogenation process according to the invention to prepare oligoamines or aminonitriles from oligonitriles, i.e. it enables full or partial hydrogenation. The extent of the full hydrogenation can be kept low if desired. A low level of byproducts are obtained, and no highly toxic substances such as cyanides are used. Expensive noble metal doping of the catalyst is not required.

### EXAMPLES

### Process Procedure:

A) Catalyst preparation: undoped Raney Ni (3g) was stirred vigorously with an aqueous solution of the desired modifier (amount as specified in table) at room temperature for 1 h. Subsequently, the catalyst was decanted, washed 2×20 ml with ethanol 2×15 ml and adiponitrile. A portion of the catalyst was used to carry out an elemental analysis in order to determine the content of modifier (see table).

Hydrogenation: 1.92 g of the ADN-moist, modified catalyst were initially charged in a 160 ml autoclave with magnet-coupled blade stirrer, electrical heater, closed-loop internal temperature control, sampling via 7 μm frit, ammonia metering via rotameter, and hydrogen sparging via the surface, and 53 g of ADN were added. 17 g of NH₃ were metered in and the autoclave was heated to 60° C. with gentle stirring (50 rpm). On attainment of this temperature, H₂ was injected to 20 bar onto the autogenous pressure of the system, which established a pressure of approx. 37 bar. At regular intervals, samples were taken which allowed the progress of the experiment to be discerned. The results of the hydrogenation experiments are listed in the table.

| | | | Metal | | | | |
|---|---|---|---|---|---|---|---|
| | | | ion | | Con- | | |
| Exam- | Modifier | | content | Time | version | ACN | HMD |
| ple | M-X | Amount | M [%] | [h] | [%] | [%] | [%] |
| 1 | Cs₂CO₃ | 69 g of | 4 | 12 | 94.4 | 63.6 | 30.1 |
| | | 13% | | | | | |
| | | solution | | | | | |
| 2 | K₂CO₃ | 69 g of | 5 | 10 | 97.8 | 52.5 | 42.3 |
| | | 13% | | | | | |
| | | solution | | | | | |
| 3 | Li₂CO₃ | 69 g of | 0.11 | 8 | 97.4 | 53.1 | 40.1 |
| | | 1% | | | | | |
| | | solution | | | | | |
| 4 | Ca(OAc)₂ | 69 g of | 0.21 | 8 | 98.3 | 49.0 | 44.0 |
| | *2H₂O | 13% | | | | | |
| | | solution | | | | | |
| 5 | Mg₂(OAc)₂ | 69 g of | 0.48 | 6 | 94.2 | 57.9 | 32.3 |
| | | 13% | | | | | |
| | | solution | | | | | |

As is evident from the examples, above-random ACN selectivities are achieved in all cases. What is meant by above-random is that, in comparison to the calculated ACN selectivity, more ACN is present at a certain conversion with the assumption that all nitrile groups are hydrogenated equally rapidly (“randomly”). Example: for 93.8% conversion, the calculated ACN selectivity is 40%; for 97.8% conversion, 26.1%.

1. A process comprising hydrogenating oligonitriles which have at least two nitrile groups in the presence of a catalyst, wherein before commencement of the hydrogenation the catalyst is pretreated by contacting with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formiates, alkaline earth metal formiates, ammonium formiate, alkali metal oxalates, alkaline earth metal oxalates and ammonium oxalate.

2. The process according to claim 1, wherein the nitrile is an alpha,omega-dinitrile.

3. The process according to claim 1, wherein the alpha,omega-dinitrile is adiponitrile.

4. The process according to claim 1, wherein all nitrile groups present in the nitrile molecule are hydrogenated to amino groups to form an oligoamine.

5. The process according to claim 1, wherein an alpha,omega-dinitrile is hydrogenated to an alpha,omega-diamine by full hydrogenation.

6. The process according to claim 1, wherein only some of the nitrile groups present in the nitrile molecule are hydrogenated to amino groups to obtain an aminonitrile.

7. The process according to claim 1, wherein an alpha,omega-dinitrile is hydrogenated to an alpha,omega-aminonitrile by partial hydrogenation.

8. The process according to claim 1, wherein the catalyst comprises at least one metal M from groups 8 to 10 of the Periodic Table.

9. The process according to claim 8, wherein the catalyst comprises, as the metal M, iron, cobalt, nickel or mixtures thereof

10. The process according to claim 1, wherein the catalyst is a nickel sponge catalyst or a cobalt sponge catalyst.

11. The process according to claim 1, wherein the catalyst comprises at least one further metal D which is selected from groups 1 to 7 of the Periodic Table.

12. The process according to claim 8, wherein the catalyst comprises, as a further metal D, at least one of the metals titanium, zirconium, chromium, molybdenum, tungsten and manganese.

13. The process according to claim 1, wherein the catalyst comprises from 0.01 to 25% by weight of alkali metal, alkaline earth metal or ammonium, based on the pretreated catalyst.

14. The process according to claim 1, wherein compound A comprises an alkali metal and the alkali metal is selected from lithium, sodium and potassium.

15. The process according to claim 1, wherein compound A comprises an alkaline earth metal and the alkaline earth metal is selected from magnesium and calcium.

16. The process according to claim 1, wherein compound A comprises alkali metals and the alkali metals are selected as phosphates or hydrogenphosphates.

17. The process according to claim 1, wherein compound A used is a mixture comprising alkali metal and alkaline earth metal compounds.

18. The process according to claim 1, wherein the catalyst is pretreated by contacting it with a solution or suspension of compound A.

19. The process according to claim 1, wherein the catalyst is contacted with an aqueous solution or suspension of compound A, the catalyst is removed and it is subsequently washed with at least one organic liquid in order to remove the water.

20. The process according to claim 1, wherein compound A is formed in situ by introducing carbon dioxide into a suspension or solution which the catalyst and a compound A* which is an alkali metal, alkaline earth metal or ammonium compound different from compounds A.

21. The process according to claim 1, wherein compound A is formed in situ by introducing ammonia into or by adding an aqueous solution of an alkali metal hydroxide or alkaline earth metal hydroxide to a suspension or solution which is the catalyst and a carboxylic acid or phosphoric acid.

22. The process according to claim 1, wherein compound A is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates.

23. The process according to claim 1, wherein compound A is selected from alkali metal carboxylates, alkaline earth metal carboxylates, ammonium carboxylates, alkali metal acetates, alkaline earth metal acetates, ammonium acetate, alkali metal formates, alkaline earth metal formates, ammonium formate, alkali metal oxalates, alkaline earth metal oxalates and ammonium oxalate.

24. The process according to claim 1, wherein compound A is selected from alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate.

25. The process according to claim 1, wherein ammonia is used additionally in the hydrogenation.

26. The process according to claim 1, wherein a solvent is used additionally in the hydrogenation.

27. An oligoamine or an aminonitrile obtained from oligonitriles by the process according to claim 1.

28. The use of catalysts as defined in claim 1 for full or partial hydrogenation of oligonitriles.

29. The use according to claim 28, wherein the catalyst is used for full hydrogenation of adiponitrile to hexamethylenediamine.

30. The use according to claim 28, wherein the catalyst is used for partial hydrogenation of adiponitrile to aminocapronitrile.

31. A catalyst comprising a metal from groups 8 to 10 of the Periodic Table which, before use, is pretreated with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formiates, alkaline earth metal formiates, ammonium formiate, alkali metal oxalates, alkaline earth metal oxalates and ammonium oxalate, excluding cobalt or nickel catalysts pretreated with alkali metal carbonates or alkali metal hydrogencarbonates.

32. The catalyst according to claim 31, wherein the catalyst comprises iron, cobalt, nickel or mixtures thereof.

33. A process for preparing the catalyst according to claim 31, which comprises treating a metal from groups 8 to 10 of the Periodic Table with a compound A which is selected from alkali metal carbonates, alkaline earth metal carbonates, ammonium carbonate, alkali metal hydrogencarbonates, alkaline earth metal hydrogencarbonates, ammonium hydrogencarbonate, alkaline earth metal oxocarbonates, alkali metal carboxylates, alkaline earth metal carboxylates, ammonium carboxylates, alkali metal dihydrogenphosphates, alkaline earth metal dihydrogenphosphates, alkali metal hydrogenphosphates, alkaline earth metal hydrogenphosphates, alkali metal phosphates, alkaline earth metal phosphates and ammonium phosphate, alkali metal acetates, alkaline earth metal acetates, ammonium acetate, alkali metal formiates, alkaline earth metal formiates, ammonium formiate, alkali metal oxalates, alkaline earth metal oxalates and ammonium oxalate, excluding cobalt or nickel catalysts pretreated with alkali metal carbonates or alkali metal hydrogencarbonates.

34. The process according to claim 33, wherein the catalyst is pretreated by contacting it with a solution or suspension of compound A.
